(19) United States
(12) Patent Application Publication
(10) Pub. No.: US 2002/0058003 A1
(43) Pub. Date: May 16, 2002

(54) ODOR CONTROL VESSEL

(76) Inventors: LEIGH E. FALKMAN, REMER, MN (US); OWEN R. ANDERSON, EDEN PRAIRIE, MN (US); DENNIS VAN DOVER, BOY RIVER, MN (US)

Correspondence Address: HUGH D JAEGER, 1000 SUPERIOR BOULEVARD, SUITE 302, WAYZATA, MN, 553911873

(21) Appl. No.: 09/166,054

(22) Filed: Oct. 5, 1998

Related U.S. Application Data
(63) Continuation-in-part of application No. 08/468,969, filed on Jun. 6, 1995, now abandoned.

(51) Int. Cl. A61L 9/00; B65D 19/00; A47B 13/08

(52) U.S. Cl. 422/275; 422/261; 422/311; 108/51.11; 108/90; 210/190

(57) ABSTRACT

A compact, portable, easily installed, self-contained, negative pressure odor control vessel for odor control possessing oxidizing properties, as well as counter-vailing and absorption properties.

### CROSS REFERENCES TO CO-PENDING APPLICATIONS

This patent application is a continuation-in-part of Ser. No. 08/468,969 entitled “Odor Control Vessel” filed on Jun. 06, 1995, by the same inventors.

### BACKGROUND OF THE INVENTION

1. Field of the Invention

The present invention is for an environmental scrubber, and more particularly pertains to an odor control vessel used for control of sulfides, mercaptans and other malodorous compounds generated in waste water collection and treatment systems and selected industrial processes.

2. Description of the Prior Art

Various odor control schemes are presently available; however, each includes one or more disadvantage. Spray atomization devices will only control odors which are directly contacted by a spray. Equipment and chemical costs for spray atomization devices are costly and somewhat inefficient. Activated carbon scrubbers will only absorb odors and have a short life. Hazardous waste problems are also associated with spent carbon. Bio-filters can create hazardous waste in spent media, and have relatively low removal efficiency. The use of deodorant blocks has also been incorporated, but was found to be very ineffective. Chemical additions were also used, but they prove to be very expensive and most are corrosive in nature. Oxidizing agents include chlorine, (chlorine/caustic) sodium and calcium hypochlorite, potassium permanganate and hydrogen peroxide. All are effective, but non-specific. Thus, they react with non-malodorous organic compounds which increases the cost of their use. As a group, these products generally pose safety problems—toxicity, the production of toxic by-products, inherent corrosive and explosive characteristics. Temperature and pH influence the effectiveness of most. Chlorine dioxide is an exception. Unwanted reaction with nitrogen-based compounds can not occur, it is much safer to handle and does not form chlorinated by-products. Clearly, what is needed is an effective and efficient reasonably priced and environmentally friendly odor scrubbing or control device, such as for large-scale applications.

### SUMMARY OF THE INVENTION

The general purpose of the present invention is an odor control vessel.

According to one embodiment of the present invention, there is provided an odor control vessel, including a compartmentized vessel including an oxidizing chamber, a polishing chamber and a sealed fan chamber. Malodorous air is drawn through an air diffuser and through an oxidizing chamber containing oxidizing media, such as, but not limited to, diatomaceous earth, and then through a diffuser plate leading to the polishing chamber containing polishing media, such as, but not limited to, diatomaceous earth, for final scrubbing of the malodorous air. A fan, which creates a negative pressure in the space from which air is being drawn, then exhausts scrubbed air to the atmosphere. The odor control vessel is constructed for ready accessibility of the interior of the odor control vessel in order to facilitate rapid change-out of scrubbing materials in the oxidizing chamber and the polishing chamber. The odor control vessel offers economic, effective and broad malodor control to address and abate community and employee odor complaints in municipal waste water treatment or other industrial processes. The odor control vessel is placed in or adjacent to a lift station, grit room, headworks, desludge chamber, press room or other enclosed point sources from which foul air is fan drawn for scrubbing in the oxidizing and polishing chamber and vented to the atmosphere.

One significant aspect and feature of the present invention is an odor control vessel incorporating the combination of oxidation, absorption and countervailing technologies to control odors.

Another significant aspect and feature of the present invention is the utilization of scrubbing media which can be recycled.

Still another significant aspect and feature of the present invention is an odor control device which utilizes a fan which is the only moving part.

An additional significant aspect and feature of the present invention is an odor control vessel, the interior of which is easily and readily accessible for renewal and/or changeout of scrubbing media.

A further significant aspect and feature of the present invention is an oxidizing media which releases chlorine dioxide.

Having thus described embodiments of the present invention, it is the principal object of the present invention to provide an odor control vessel.

### BRIEF DESCRIPTION OF THE DRAWINGS

Other objects of the present invention and many of the attendant advantages of the present invention will be readily appreciated as the same becomes better understood by reference to the following detailed description when considered in connection with the accompanying drawings, in which like reference numerals designate like parts throughout the figures thereof and wherein:

FIG. 1 illustrates an isometric view of an odor control vessel, the present invention;

FIG. 2 illustrates an exploded view of the odor control vessel; and,

FIG. 3 illustrates a cross sectional side view of the odor control vessel.

### DETAILED DESCRIPTION OF THE PREFERRED EMBODIMENTS

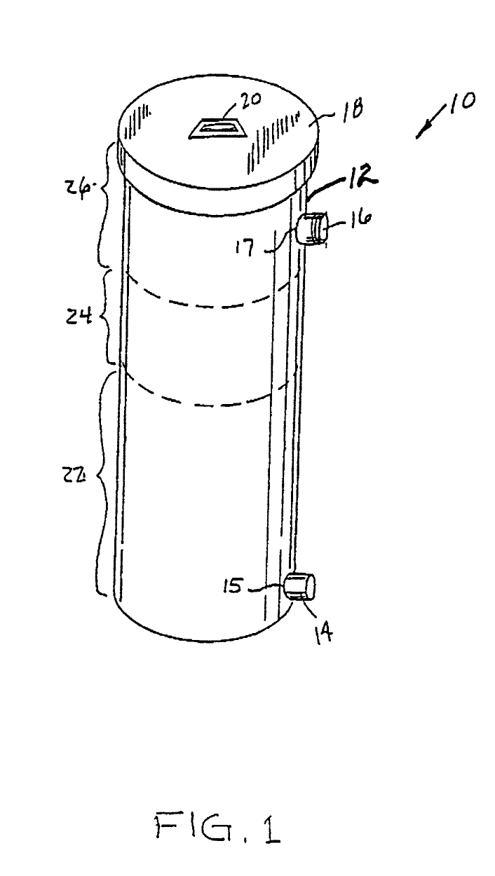

FIG. 1 illustrates an isometric view of an odor control vessel 10, the present invention, for removal of hydrogen sulfide and other pungent, foul or otherwise offensive odors and smells from an enclosed area. Visibly illustrated components include a cylindrically shaped vessel 12 of plastic or other suitable material. A foul air intake tube 14 passes through a hole 15 at the bottom region of the vessel 12, and a clean air exhaust tube 16 passes through a hole 17 the upper region of the vessel 12. A vented cover 18 with a handle 20 aligns over and about the upper edge of the vessel 12. The odor control vessel 10 divides into an oxidizing chamber 22 located in the lower region of the vessel 12, a polishing chamber 24 located in the upper mid-region of the vessel 12 and a fan chamber 26 located in the upper region of the vessel 12.

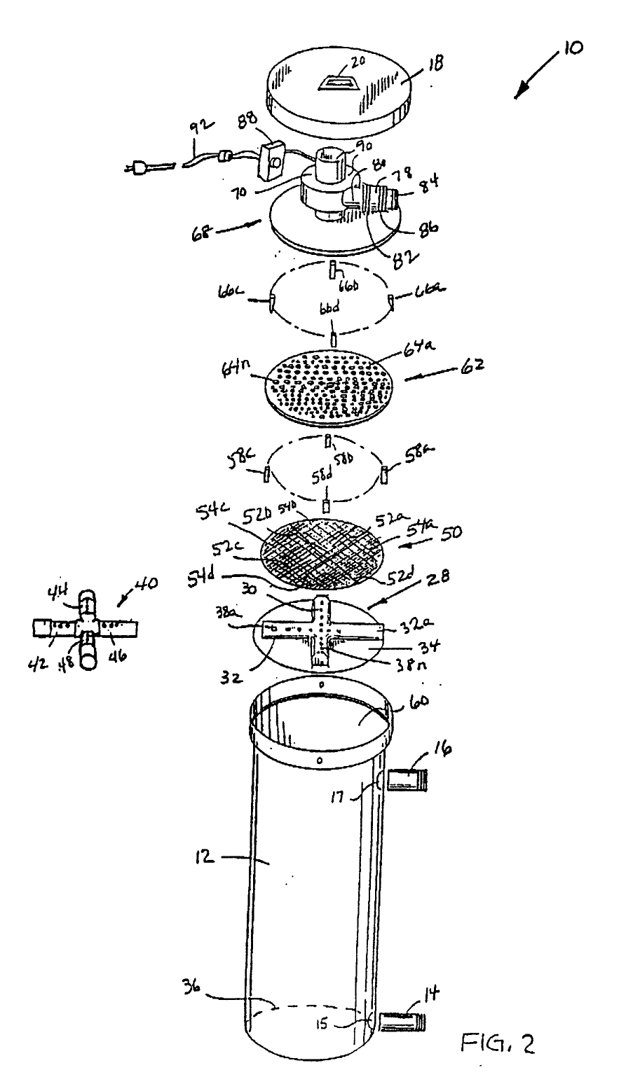

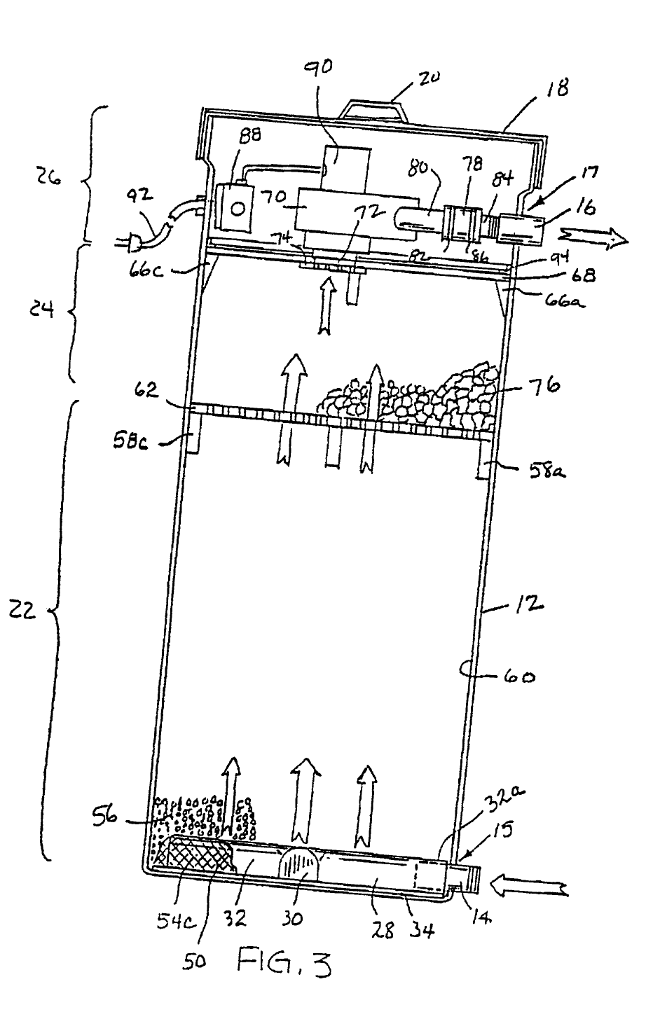

FIG. 2 illustrates an exploded view of the non-chemical components of the odor control vessel 10 where all numerals correspond to those elements previously described. A one-piece molded air diffuser 28 includes intersecting open bottom manifold members 30 and 32 aligned and contiguous with a circular disk 34. The circular disk 34 aligns to the bottom 36 of the vessel 12 to fully form the remaining portion of the manifold members 30 and 32. End 32a of manifold 32 plumbs to the inner end of the foul air intake tube 14 as illustrated in FIG. 3. A plurality of vent holes 38a-38n are located along the upper curved areas of the intersecting manifold members 30 and 32. An alternate air diffuser 40 having intersecting tubular manifold members 42, 44, 46 and 48 is illustrated to the left of air diffuser 28. The alternate air diffuser 40 can be interchanged with the one-piece molded air diffuser 28. A circular flexible plastic screen 50, having slits 52a-52d, divides the screen 50 into pie shaped segments 54a-54d, and aligns over and about the intersecting manifold members 30 and 32 of air diffuser 28 to prevent entry of oxidizing media, such as stabilized oxidizing media 56 of FIG. 3, into the vent holes 38a-38n. The oxidizing media will be further described in FIG. 3. Slits 52a-52d allow the pie shaped segments 54a-54d to deform and to conform to the general shape of the intersecting manifold members 30 and 32 to effect an effective screen seal of the vents 38a-38n. A plurality of supports 58a-58d secure appropriately about the interior wall 60 of the vessel 12 and at the upper region of the oxidizing chamber 22 to support a middle air diffuser plate 62 which includes a plurality of diffuser holes 64a-64n of any suitable size. The holes can be covered with an optional polymer mesh or screen. The middle air diffuser plate 62 separates oxidizing chamber 22 from the polishing chamber 24, as illustrated in FIG. 3. A plurality of wedge shaped supports 66a-66d secure appropriately about the interior wall 60 of the vessel 12 and at the upper region of the polishing chamber 24 to support the disk shaped blower chamber fan support member 68. The wedge shape of the supports 66a-66d allows for easier tipping insertion or removal of the diffuser plate 62 due to the less restrictive geometry of the wedge shaped support members 66a-66d. A fan 70 mounts to the fan support member 68. As illustrated in FIG. 3, the intake 73 of fan 70 aligns to a hole 72 in the fan support member 68. A plate 74, having a plurality of orifices, covers the bottom of the hole 72 to prevent entry of polishing media 76 into the fan 70. A flexible rubber coupling hose 78 secures over the output 80 of the fan 70 by a clamp 82. A pipe member 84, having a threaded outboard end, secures in the other end of the flexible rubber coupling hose 78 by a clamp 86. The threaded end of pipe member 84 screwingly engages the interior of the clean air exhaust 16. Decoupling of the fan 70 and fan support member 68 for replacement of treated media is readily accomplished by loosening clamp 86 and unscrewing the pipe member 84 from the interior threads of the pipe member 84. The fan 70 and fan support member 68, in addition to the diffuser plate 62, are then removed for media renewal. An electric fan speed control 88 connects to the fan motor 90 and includes cord 92.

### MODE OF OPERATION

FIG. 3, a cross sectional side view of the odor control vessel 10, best illustrates the mode of operation where all numerals correspond to those elements previously described. A seal 94 aligns about the inner surface 60 of the vessel 12 to effect a seal between the fan support member 68 and the inner surface 60. Seal 94 effectively seals the fan chamber 26 from the chemically treated oxidizing media 56 in the oxidizing chamber 22 and the polishing media 76 in the polishing chamber 24. Air flow through the odor control vessel 10 is indicated by arrows starting at the foul air intake tube 14 which pass through the air diffuser 28, oxidizing media 56 in the oxidizing chamber 22, diffuser plate 62, polishing media 76 in the polishing chamber 24, perforated plate 74, fan 72, fan output 80, coupling hose 78, pipe member 84, and finally through the clean air exhaust 16 where the scrubbed air is vented to the atmosphere. The forced air fan is optional, and air flow can be passive or natural, therefore not requiring a fan. The use of a controlled release chlorine dioxide oxidizing media 56, such as, but not limited to, Persnickety Ox Pellets, Appendix A, available from Syneco Systems, Inc. in St. Louis Park, Minn., in conjunction with polishing chamber media 76, such as, but not limited to, Countervailant™ impregnated diatomaceous earth, provides for safe, extremely effective broad spectrum malodor control. Referenced percentages of W/V available, chlorine dioxide can range from 4.5% to 11.5% and a cubic foot of the media can range from 2.48 to 6.34 pounds of chlorine dioxide.

The polishing media 76 releases Countervailant™ chemistry. The oxidizing chamber media 56 releases chlorine dioxide which is impregnated into diatomaceous earth which is amorphous silica containing less than 1% of crystalline silica. The exact size, shape and density of medias 56 and 76 is determined by the needs of the specific odor control vessel 10. These needs include a controlled and determined surface area exposed relative to the residence time of malodorous air in each chamber.

The size, shape and density of the medias 56 and 76 is also determined by the size of the containment chamber, such as oxidizing chamber 22 and polishing chamber 24. The dimensions requirements of medias 56 and 76, for purposes of illustration and example, change with the sizes of oxidizing chamber 22 and the polishing chamber 24. Larger sized oxidizing and polishing chambers 22 and 24, respectively, require large sized oxidizing chamber media 56 and polishing chamber 76, and smaller sized units require smaller sized oxidizing chamber media 56 and polishing chamber 76. The crushing effect to lower layers of media 56 and 76 in larger chambers 22 and 24 require larger sized media 56 and 76 to resist such effects, smaller sized units may use smaller sized media 56 and 76 because the crushing effect is less, and smaller sized media optimizes available surface area exposed to the malodorous air being drawing through it.

The chlorine dioxide-releasing oxidizing chamber media 56 incorporated in the oxidizing chamber 22 provides for an extremely powerful oxidizing agent. For example, one pound of chlorine dioxide is equivalent in oxidation power to 3.8 pounds of potassium permanganate. Because chlorine dioxide reacts selectively and primarily with only the most offensive odor producing compounds (hydrogen sulfide [H₂S], organic sulfurs, organic amines, petroleum distillates), the odor control vessel 10 system will normally provide a much longer service life than other devices, such as activated carbon. Similarly, chlorine dioxide will remain intact longer than chlorine, hypochlorite, peroxide and permanganate. Unwanted reactions do not occur. Power is not needlessly spent. Chlorine dioxide remains available and ready on demand.

The polishing chamber 24 protects against the escape of malodors, such as ammonia, which cannot be oxidized by chlorine dioxide. The Countervailant™ treated media 76 in chamber 24 allows other barriers to be built against the escape of malodors which cannot be readily oxidized.

Countervailant™ technology is highly specialized, complex and broadly useful chemistry. It is especially effective on non-oxidizable odors, such as ammonia, and incorporates neutralization technology, but expands considerably beyond it. Polymeric absorption is a facet which involves the building up malodor molecules via electrostatic attractions and Van der Waals forces so that they are not recognized as malodors. The process of esterification is also incorporated. Acids and alcohols react to form esters. These esters normally have a pleasant scent. Countervailant™ technology is effective in dealing with malodors in both liquid and gaseous phases.

Appendix 1 recites an oxidizing air scrubber and is attached hereto as Appendix 1.

Various modifications can be made to the present invention without departing from the apparent scope hereof.

[image]

[image]

[image]

[image]

[image]

It is claimed:

1. An odor control vessel system comprising, in order:
a. air intake;
b. an oxidizing chamber;
c. a polishing chamber; and,
d. an air exhaust.

2. The vessel of claim 1, including a fan chamber with a fan and power control means for controlling said fan.

3. An odor control vessel system comprising, in order:
a. air intake;
b. an oxidizing chamber with an oxidizing material;
c. a polishing chamber with Countervailant™ chemistry; and,
d. an air exhaust.

4. The vessel of claim 3, wherein said material is a granular solid material.

5. The vessel of claim 3, including a fan chamber with a fan and a power control means for controlling said fan.

6. A process for controlling odor comprising the steps of:
a. intaking air from an odor area;
b. passing the air through an oxidizing chamber;
c. passing the air through a polishing chamber; and,
d. exhausting the air.
